(19) United States
(12) Patent Application Publication
(10) Pub. No.: US 2026/0144937 A1
(43) Pub. Date: May 28, 2026

(54) APPLICATOR, DEVICE AND METHOD FOR NEEDLELESS INJECTION OF AN ACTIVE MEDIUM FOR INJECTION

(71) Applicant: TRANSDERMJET GMBH, Wien (AT)

(72) Inventors: Jürgen STOCKMAR, Wien (AT); Peter EMMINGER, Wien (AT); Guntram SCHNETZ, Biedermannsdorf (AT)

(21) Appl. No.: 19/122,683

(22) PCT Filed: Oct. 13, 2023
(86) PCT No.: PCT/EP2023/078450

§ 371 Date: Sep. 22, 2025

(30) Foreign Application Priority Data
Oct. 19, 2022 (AT) A50808/2022

(51) Int. Cl. A61M 5/30; A61M 5/145

(52) U.S. Cl. CPC A61M 5/30; A61M 5/14526; A61M 5/1456

(57) ABSTRACT

For an applicator for needleless injection of an active medium (WM), which permits a high injection pressure and ensures a reliable and secure separation of the injectable active medium (WM) from the actuation of the device (1), an injection valve (20) is provided in the applicator (B) and a working medium (AM) with a working pressure (p_{A}) can be applied at an inlet (27) of the injection valve, a pressure volume (21) is provided in the applicator (B), and the pressure volume (21) is filled with working medium (AM) at an initial pressure less than the working pressure (p_{A}), the operational injection valve (20) is configured to inject into the pressure volume (21) a predetermined injection quantity of the working medium (AM) with an injection pressure (p_{E}) between the working pressure (p_{A}) and the initial pressure, and there is provided in the applicator (B) a movably arranged plunger (22), which at a first end has a plunger piston (26) that at least partially delimits the pressure volume (21), and which, with an opposite second end, bears on the pressure piston (25) such that the operational plunger (22) is movable in the direction of the pressure piston (25) in order to move the pressure piston (25) so as to deliver an active-medium pulse from at least nozzle opening (29)m, (FIG. 2).

The present invention relates to an applicator for needleless injection of an injectable active medium into a body surface with a nozzle unit with a nozzle volume into which the injectable active medium is filled, wherein, on the nozzle unit, at least one nozzle opening is provided, which is connected to the nozzle volume, and wherein, in the nozzle unit, a pressure piston is arranged, which at least partially delimits the nozzle volume and which is movably arranged in the nozzle unit. The invention also relates to a method and a device for needleless injection of an injectable active medium into a body surface using such an applicator.

WO 2012/139774 A1 describes a device for needleless injection of a medium into or under the skin with a series of pressure pulses. The device defined there is characterized by the lowest construction costs for achieving the described purpose. Due to the minimally invasive needleless injection into or through the skin, this device falls into hazard class IIb according to EU Regulation 2017/745 on medical devices. Therefore, all surfaces in the device that come into contact with the injectable medium must be biocompatible and sterilizable. Available standard parts such as high-pressure pumps, valves, accumulators, or hoses usually do not meet this requirement, which is why it is difficult to put such a device on the market. Special developments would be necessary for individual components of such a device, which would make the device complex and expensive.

EP 2 957 309 B1 describes a cylinder-piston unit for a needleless injector. The injectable medium is contained in a cylinder. The cylinder has a nozzle at one end for discharging the medium and the piston at the opposite end, which is moved in the direction of the nozzle in a pulsed manner by an actuating means not described in detail, whereby the medium is delivered through the nozzle into the patient's skin.

EP 2 035 065 B1 describes a device for the needleless injection of an aqueous medium into the skin of a patient. In a nozzle filled with the injectable medium, a piston is moved back and forth by a pneumatic cylinder. During the backward stroke of the piston, the aqueous medium is conveyed into the nozzle via a dosing unit, which medium is discharged via the nozzle during the forward stroke of the piston. However, only pressures of approx. 150 bar are possible with this device, which is too low for many applications, especially with higher viscosity media. Apart from this, air can also be sucked in via the nozzle during the backward stroke of the piston, which can impair the discharge of the medium during the next forward stroke of the piston. KR 20140054812 A also discloses such a device.

It is an object of the present invention to provide an applicator for needle-free injection of an active medium, which permits a high injection pressure and ensures reliable and safe separation of the injectable active medium from the actuation of the device.

This object is achieved in that an injection valve is provided in the applicator, to which a working medium at a working pressure can be applied on the inlet side, a pressure volume is also provided in the applicator, and the pressure volume is filled with working medium at an initial pressure less than the working pressure, the injection valve is set up to inject a predetermined injection quantity of the working medium into the pressure volume during operation at an injection pressure between the working pressure and the initial pressure, and there is provided in the applicator a movably arranged plunger, which at a first end has a plunger piston that at least partially delimits the pressure volume, and which, with an opposite second end, bears upon the pressure piston, such that the operational plunger is movable in the direction of the pressure piston in order to move the pressure piston so as to deliver an active-medium pulse from the at least one nozzle opening.

The active medium and the working medium are physically and spatially separated by the plunger. In addition, only a very low pressure (usually atmospheric pressure) acts in the pressure volume compared to the working pressure. Contamination of the active medium by the working medium is excluded if the applicator is used correctly. This also reliably separates the active medium in the nozzle unit from the actuation, because the active medium is not accessible at any point outside the nozzle unit and in particular not inside the applicator.

It is also advantageous if the working pressure of the working medium is generated separately from the applicator. For this purpose, a base unit can be provided which supplies the working medium at the working pressure, and the base unit is connected to the applicator via a pressure hose, in order to supply the working medium at the working pressure to the applicator. None of the components of the base unit come into contact with the active medium, which means that no particular requirements are placed on these components; in particular, they do not have to be biocompatible or sterilizable.

Preferably, the nozzle unit is detachably arranged on the applicator. This allows the active medium to be replaced quickly and easily.

Advantageously, the nozzle unit is designed with a cartridge holder with an internal recess, and an active-medium cartridge is interchangeably inserted in the internal recess, wherein the at least one nozzle opening is provided on the active-medium cartridge, and the pressure piston is movably arranged in the active-medium cartridge. The active-medium cartridge, with different active media and also with different active-media volumes, can be provided as operating material for the operation of the applicator. The active-medium cartridge can be easily and safely replaced in the applicator.

In order to be able to place the applicator in a defined position on the body surface, a tubular spacer is advantageously provided on the active-medium cartridge in the region of the nozzle opening, which spacer protrudes by a predetermined length axially from the active-medium cartridge and which surrounds the nozzle opening so that the active-medium pulse exits through the tubular spacer. This ensures that the distance between the nozzle opening and the body surface is set and maintained. The tubular spacer also prevents splashing of the active medium.

It is particularly advantageous that the inner recess in the cartridge holder and the active-medium cartridge converge in a conical shape in the direction of the at least one nozzle opening. The active-medium cartridge can be shaped in the opposite direction. In this way, the active-medium cartridge can be pressed into the cartridge holder when in use so that the active-medium cartridge lies securely on and, above all, over the entire surface of the cartridge holder. This prevents the active-medium cartridge from expanding or deforming during operation of the applicator, which could impair the pressure build-up in the nozzle volume of the active-medium cartridge and thus the generation of the active-medium pulse.

In order to be able to fill the nozzle volume safely and easily with active medium, the pressure piston can be designed with an axially continuous hole. This allows filling to take place through the pressure piston and prevents air from being trapped in the nozzle volume during filling. For this purpose, a filling connection is advantageously provided on the side, facing away from the at least one nozzle opening, of the pressure piston in order to fill active medium into the nozzle volume via the filling connection and the axially continuous recess. A filling element can be connected to the filling connection for filling.

Preferably, a control button is provided on the applicator to actuate the injection valve. The control button can be actuated by an applicating person.

The applicator can also be used to inject a predetermined injection quantity of the working medium into the pressure volume several times during operation via the injection valve at an injection pressure between the working pressure and the initial pressure. This means that several active-medium pulses are emitted in succession. Repeated injection can be controlled by the applicating person, but can also take place at predetermined intervals.

It is advantageous if the injection valve varies the injection pressure during the delivery of at least one active-medium pulse. This allows pressure modulation to be achieved, and the injection of the active medium into the body surface can be controlled more flexibly, which also makes possible gentler application. In this context, it is particularly advantageous if the injection valve first injects at a first injection pressure for a first period of time and then injects at a second injection pressure for a second period of time, wherein the second injection pressure is lower than the first injection pressure. The body surface can be penetrated at the first high pressure, and then a desired quantity of active medium can be injected into the body surface at a lower, gentler pressure.

To generate the working pressure, a pump is advantageously provided in the base unit, which increases the pressure of a working medium from a storage tank to the working pressure, wherein the pump delivers into a pressure line that is connected to the pressure hose.

In the base unit, a return line preferably branches off from the pressure line downstream of the pump, wherein a controllable return valve is arranged in the return line in order to depressurize the pressure line via the return line when the return valve is open. In this way, the device and the applicator can be easily depressurized—for example, to remove the nozzle unit, e.g., to replace an active-medium cartridge. In the depressurized state, the plunger can also simply be pushed back into its initial position, preferably automatically by a compression spring acting upon the plunger.

The compression spring preferably engages with a first end on the side, facing away from the pressure volume, of the plunger piston, and a second end of the compression spring bears preferably upon a part, immovable relative to the plunger, of the applicator, which allows a structurally simple design.

To increase operational safety, a safety switch is provided advantageously on the device to enable or disable operation of the applicator. Only when the safety switch is activated can the device be actuated using a control button. This can prevent the erroneous delivery of active-medium pulses at the applicator.

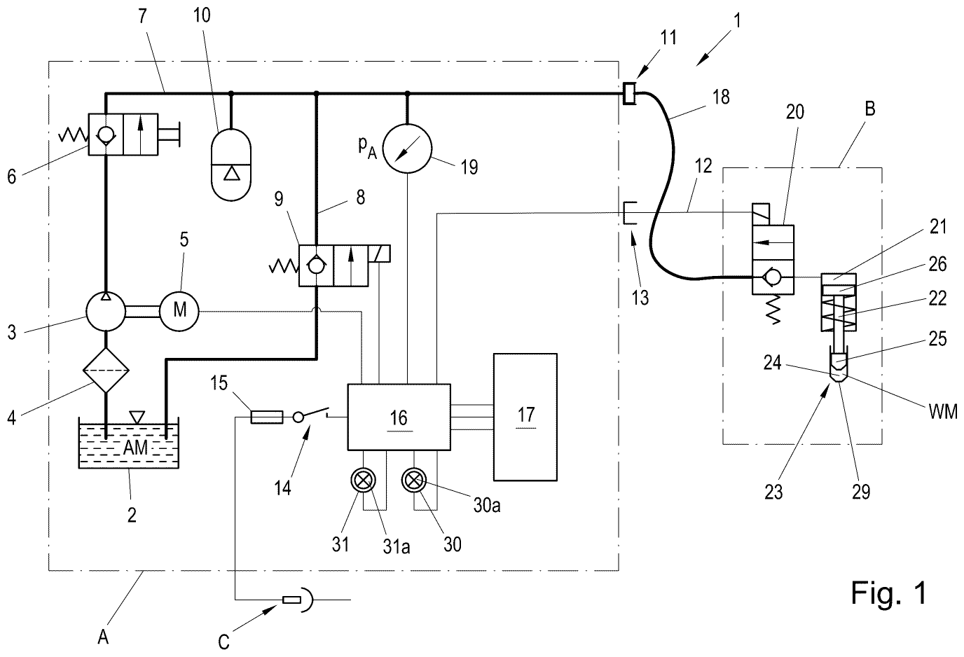

The present invention is explained in more detail below with reference to FIGS. 1 to 7, which show exemplary, schematic, and non-limiting advantageous embodiments of the invention. In the figures:

FIG. 1 is a schematic representation of the device according to the invention with base unit and applicator,

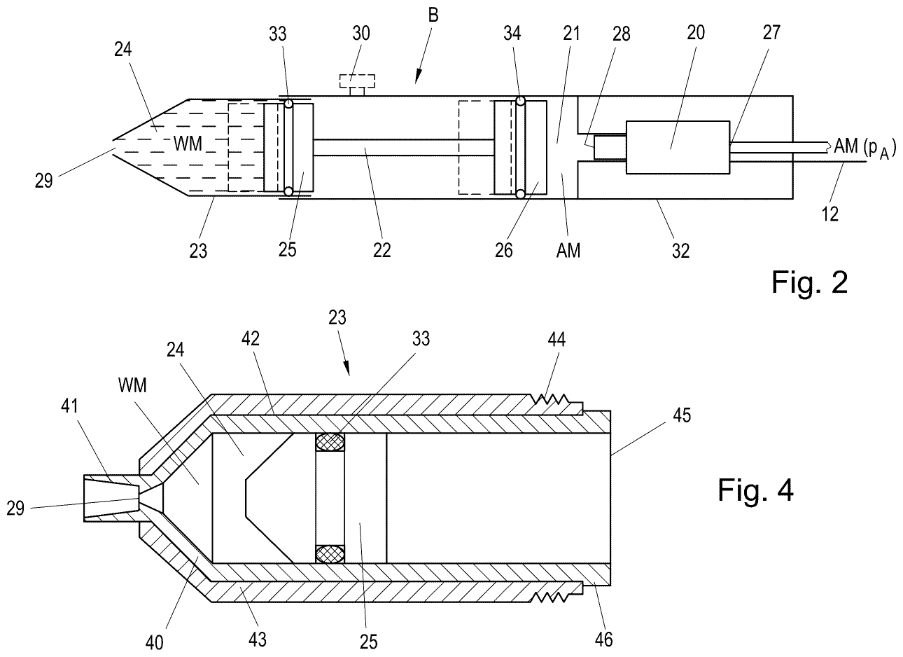

FIG. 2 shows an embodiment of an applicator according to the invention,

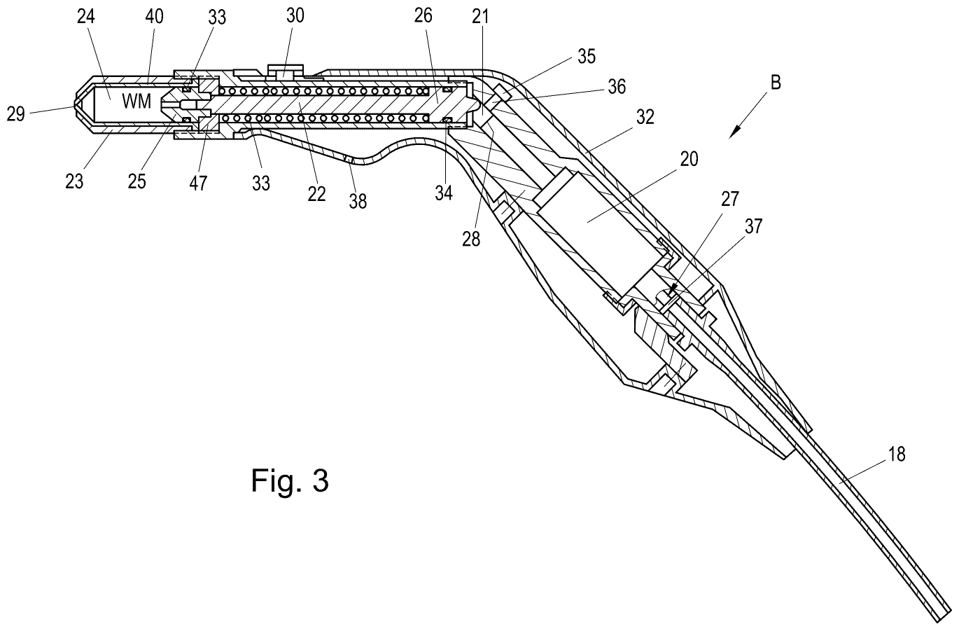

FIG. 3 shows a further embodiment of an applicator according to the invention,

FIG. 4 shows an embodiment of a nozzle unit for an applicator according to the invention,

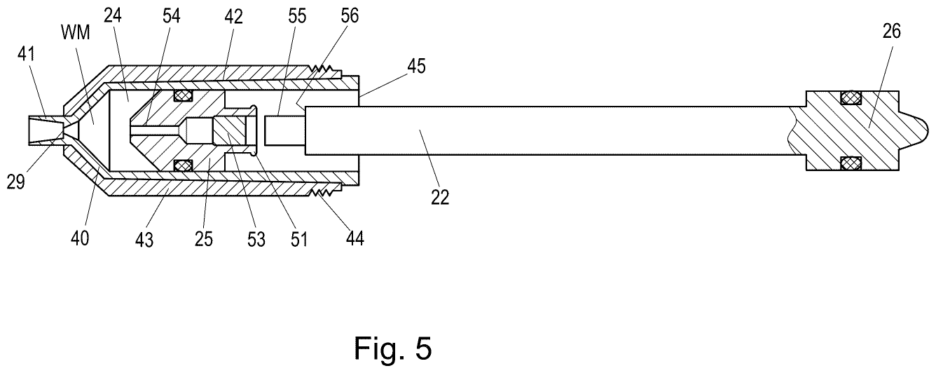

FIG. 5 shows a further embodiment of a nozzle unit for an applicator according to the invention,

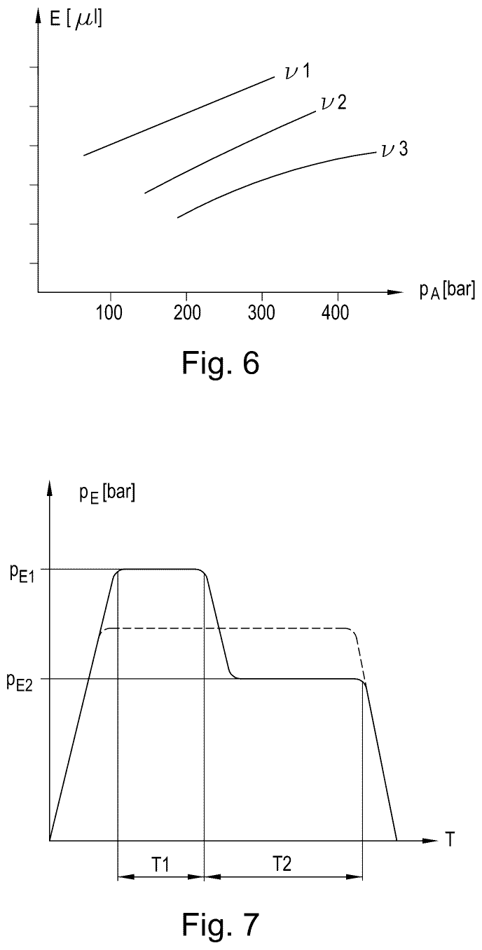

FIG. 6 shows an exemplary correlation between working pressure and injection quantity for different active media, and

FIG. 7 shows an example of pressure modulation of the pressure in the pressure volume.

The device 1 according to the invention is used for the needleless injection of various active media WM into a body surface of a patient (human or animal)-for example, the skin or a fingernail or toenail. Examples of active media WM include aqueous solutions of disinfectants, antibiotics, botulinum toxin, and anesthetics, as well as highly viscous hyaluronic acids with kinematic viscosities of up to 1m²/s (1 million cSt), platelet-rich plasma (PRP), suspensions with particles with maximum particle dimensions of less than half the cross-sectional area of the nozzle, or antifungal agents (e.g., against nail fungus).

The device 1 according to the invention separates the generation and regulation of the working pressure p_{A} of the working medium AM required for the described function in a base unit A from the parts required for the intermittent, needleless injection of the injectable active medium WM in an applicator B. This means that mainly standard components can be used for the working medium AM in the base unit A and in the applicator B. This also ensures compliance with EU Regulation 2017/745.

The device 1 therefore consists of a base unit A and an applicator B. The base unit A is used to generate and regulate the working pressure p_{A} of the working medium AM. The applicator B is used for needleless injecting and is held or guided by the applicating person (e.g., a doctor). The applicator B is connected to the base unit A via a pressure hose 18. For this purpose, a suitable media coupling 11, e.g., a quick-release coupling, can be provided on the base unit A and/or on the applicator B in order to connect the pressure hose 18 in a detachable manner.

An electrical control line 12 is also provided between the base unit A and the applicator B to permit a communication connection between the applicator B and the base unit A-for example, for data exchange or to control the functions of the applicator B. A suitable connector 13, e.g., a socket plug connection, can also be provided on the base unit A and/or on the applicator B for this purpose in order to connect the control line 12 detachably.

The base unit A can therefore also be used with various applicators B.

In a preferred embodiment, the control line 12 and/or the pressure hose 18 are permanently connected to the applicator B. A media coupling 11 and/or a connector 13 are provided on the base unit A in order to connect the pressure hose 18 and/or the control line 12. This means that the applicator B can be easily separated from the base unit A or replaced.

The control line 12 and the pressure hose 18 can also be guided in a common hose package, which can facilitate the handling of the applicator B.

The base unit A is supplied with electrical energy from a power supply C, e.g., via a mains connection as shown in FIG. 1 or via a battery in the base unit A or a battery that can be connected to the base unit A.

The base unit A can have a main switch 14, for switching the device 1 on and off, and an electrical fuse 15.

A control unit 16 is provided in the base unit A. An input/output unit 17, e.g., a touchscreen, a display, lamps, lights, buttons, handwheels, sliders, etc., can also be provided on the base unit A in order to display information to the applicating person and/or to permit the applicating person to enter data to control functions of the device 1.

The control unit 16 is usually microprocessor-based hardware running control software. However, the control unit 16 can also be designed as an integrated circuit, such as a field programmable gate array (FPGA) or application-specific integrated circuit (ASIC). Combinations are of course also conceivable.

To generate pressure for the working medium AM, preferably a synthetic fluid or medical silicone oil, a storage tank 2 is provided in the base unit A, from which a pump 3 draws working medium AM in order to increase the pressure of the working medium AM to the desired working pressure p_{A}. The pump 3 conveys the working medium AM into a pressure line 7. A filter 4 can be provided between the pump 3 and the storage tank 2. The pump 3 can be driven by an electric motor 5, which can be controlled by the control unit 16.

The pump 3 can be designed as a speed-controlled pump in order to set a specific working pressure p_{A} of the working medium AM in the pressure line 7.

To set the working pressure p_{A} of the working medium AM, a pressure control valve 6 can also be provided downstream of the pump 3, as in the embodiment shown in FIG. 1. The pressure control valve 6 can be controlled electrically, e.g., by the control unit 16, or it can be controlled mechanically—for example, via a handwheel on the base unit A. A desired working pressure p_{A} is set on the pressure control valve 6 via the actuation. A current working pressure p_{A} in the pressure line 7 could also be displayed on a pressure indicator—for example, on a pressure gauge or on the input/output unit 17.

A pressure sensor 19 can also be provided in the pressure line 7 in order to detect a current working pressure p_{A}. The detected working pressure p_{A} can be signaled to the control unit 16 and/or can be displayed on the input/output unit 17.

In the base unit A, a return line 8 can also be provided, which branches off from the pressure line 7 and leads into the storage tank 2 via a return valve 9. The return valve 9 can be controlled by the control unit 16 (as in FIG. 1) to open the return line 8 into the storage tank 2—for example, to depressurize the device 1. Alternatively, the return valve 9 can also be actuated manually. In order to depressurize the pressure volume 21 as well, the injection valve 20 can be opened-for example, by activating it via the control unit 16. This makes it easier to remove the nozzle unit 23.

Furthermore, a pressure accumulator 10 can also be provided in the base unit A, which accumulator is connected to the pressure line 7. The pressure accumulator 10 ensures that the working pressure p_{A} in the pressure line 7 is as constant as possible and is intended to suppress pressure fluctuations.

When the pressure hose 18 is connected, the pressure line 7 is connected to it in order to supply the working medium AM to the applicator B at the set working pressure p_{A}.

At least some of the components and parts described above are preferably arranged in a sealed housing of the base unit A.

However, the embodiment of the base unit A as shown according to FIG. 1 is only an example and can be designed in any other way. The only essential feature of the invention is that a working medium AM at a desired working pressure p_{A} is provided by the base unit A, which is guided to the applicator B via the pressure hose 18. The working pressure p_{A} of the working medium AM can typically be up to 500 bar, which is sufficient for most treatments.

The structure of the applicator B ensures reliable and safe separation of the working medium AM and the injectable active medium WM. FIG. 2 shows the structure of the applicator B in schematic form.

The applicator B according to the invention comprises a controlled injection valve 20, a pressure volume 21, a plunger 22, and a nozzle unit 23.

An injection valve 20 is a controlled valve which, when actuated, forwards a working medium AM present at an inlet 27 of the injection valve 20 at a working pressure p_{A} to an outlet 28 of the injection valve 20. The pressure hose 18 carrying the working medium AM at the working pressure p_{A} is connected to the inlet 27 of the injection valve 20 when the applicator B is used. If the injection valve 20 is not actuated, no working medium AM is transferred from the inlet 27 to the outlet 28.

The injection valve 20 is preferably electrically actuated and can be connected to the control line 12, via which the injection valve 20 can be controlled for actuation-for example, by the control unit 16 in the base unit A.

In the nozzle unit 23, a nozzle chamber 24 is provided, in which the injectable active medium WM is arranged. The nozzle chamber 24 is connected to a nozzle opening 29 of the nozzle unit 23 in order to eject the injectable active medium WM via the nozzle opening 29 during operation. The nozzle chamber 24 is delimited, preferably on the opposite side of the nozzle opening 29, by a pressure piston 25. The pressure piston 25 is movably arranged in the nozzle chamber 24. A sealing element 33, e.g., an O-ring, is provided between the pressure piston 25 and the wall of the nozzle unit 23 surrounding the nozzle chamber 24, or the active-media cartridge 40 (see, for example, FIG. 4 or FIG. 5), in order to prevent the active medium WM from escaping between the pressure piston 25 and the wall of the nozzle unit 23, or the active-medium cartridge 40 (see, for example, FIG. 4 or FIG. 5). When the device 1 is actuated, the pressure piston 25 reduces the nozzle chamber 24 in order to eject the injectable active medium WM from the nozzle opening 29.

The nozzle opening 27 typically has a diameter of 0.1 to 0.3 mm, depending upon the viscosity of the active medium WM.

The nozzle unit 23 with the pressure piston 25 is preferably detachably and interchangeably arranged on the applicator B. For example, the nozzle unit 23 can, via a screw thread, be screwed onto the applicator B or screwed into the applicator B.

The applicator B can have an applicator housing 32 in which the injection valve 20, the pressure volume 21, and the plunger 22 are arranged. The nozzle unit 23 is detachably arranged on the applicator housing 32—for example, screwed into the applicator housing 32.

The plunger 22 bears with one end upon the pressure piston 25 or is connected to the pressure piston 25 at this end in order to move the pressure piston 25 during operation of the applicator B. At the other end of the plunger 22, a plunger piston 26 is provided, which is adjacent to the pressure volume 21 and at least partially delimits the pressure volume 21. A sealing element 34, e.g., an O-ring, is provided between the plunger piston 26 and the wall, surrounding the pressure volume 21, of the applicator B in order to prevent the working medium AM from escaping between the plunger piston 26 and the wall of the applicator B.

The pressure volume 21 is filled with the working medium AM, preferably without air inclusions that could have a pressure-damping effect. The pressure volume 21 can be filled with the working medium AM before the applicator B is put into operation.

The injection valve 20 is supplied with working medium AM at working pressure p_{A} on the inlet side at inlet 27 via pressure hose 18. The outlet 28 of the injection valve 20 is connected to the pressure volume 21. If the injection valve 20 is actuated and thus opened, a defined injection quantity of the working medium AM is injected into the pressure volume 21 at the working pressure p_{A}.

Injecting the working medium AM at a high working pressure p_{A} into the pressure volume 21 creates a short pressure pulse in the pressure volume 21. The plunger 22 is moved thereby via the plunger piston 26 in a pulsed manner in the direction of the pressure piston 25 (indicated by a dashed line and greatly exaggerated in FIG. 2), which in turn moves the pressure piston 25, which moves freely in the nozzle chamber 24, and the injectable active medium WM is ejected from the nozzle opening 29 in a pulsed manner, at high pressure and at high speed in the form of an active-medium pulse with a specific injection quantity E. The movement of the plunger 22 increases the pressure volume 21 by the injection quantity, as a result of which the pressure pulse in the pressure volume 21 is quickly reduced due to the incompressibility of the working medium AM, and the pressure in the pressure volume 21 quickly reaches the initial pressure acting in the pressure volume 21, so that the movement of the pressure piston 25 is also quickly terminated. The initial pressure in the pressure volume 21 is always lower, usually lower by several powers of ten, than the working pressure p_{A}. The initial pressure is usually atmospheric pressure, and the working pressure p_{A} is in the range of several tens to several hundred bar.

The working pressure p_{A} of the working medium AM applied upstream of the injection valve 20 thus propagates via the open injection valve 20 to the plunger piston 26 and presses the plunger 22 with the plunger piston 26 in the direction of the nozzle unit 23 by the amount resulting from the injection quantity. The plunger 22 transmits this movement to the pressure piston 25 in the nozzle unit 23 and forces the active medium WM filled in the nozzle volume 24 out of the at least one nozzle opening 29 at a speed of up to 250 m/s. When escaping the nozzle opening 29, the active medium WM forms a micro-jet with high kinetic energy, which penetrates the body surface and penetrates to a certain depth.

The outlet pressure of the active medium WM from the nozzle unit 23 is of course dependent upon the working pressure p_{A} and also substantially depends upon pressure losses in the applicator B, e.g., in the injection valve 20, friction losses, flow losses, etc. However, the outlet pressure can also be influenced by the ratio of the piston surfaces of the plunger piston 26 and of the pressure piston 25.

The injection quantity E substantially results from the working pressure p_{A}, the viscosity v of the active medium WM, the flow area when the injection valve 20 is open, and the opening time t during which the injection valve 20 is open. This correlation can be determined empirically for a specific applicator B and can be assumed to be known.

FIG. 6 shows an example of the correlation between working pressure p_{A} and injection quantity E, e.g., in microliters, at different viscosities v of the active medium WM and for a specific opening time t of the injection valve 20. The curves show the course of the injection quantity E at different viscosities v of the active medium WM as a function of the working pressure p_{A}. In this example, the viscosity increases from v1 to v3. Such correlations can be determined for different opening times t. It is possible to interpolate therebetween as required. Such correlations can also be created for different skin types.

This correlation, or such correlations, can be stored in the control unit 16-for example, in tabular form. This allows the opening time t of the injection valve 20 to be determined for a specific active medium WM and a specific working pressure p_{A}, with which the injection valve 20 must be controlled in order to deliver a specific injection quantity E with the applicator B. This correlation can also be used to obtain an advantageous working pressure p_{A} for a specific active medium WM for specific skin types.

Typical opening times of the injection valve 20 are in the range of 2 to 80 ms, advantageously between 5 and 40 ms.

The injection of the working medium AM into the pressure volume 21 by means of the injection valve 20 can take place once, whereby a single emitted “spurt” (active-medium pulse) of the injectable active medium WM is effected via the nozzle unit 23. The active-medium pulses can be repeated as required, whereby an active-medium pulse is emitted each time device 1 is actuated. This can be controlled by the applicating person. However, the working medium AM can be injected in a “burst mode” when the device 1 is actuated at predetermined time intervals in order to cause several successive “ ” (active-medium pulses) of the injectable active medium WM. Intermittent operation with successive active-medium pulses is achieved thereby. In burst mode, time intervals on the order of a hundred milliseconds to one second, e.g., between 0.5 and 2 seconds, are typical. This can be controlled via the control unit 16—for example, in response to the applicating person's input via the input and output unit 17. Burst mode can be maintained as long as the device 1 is actuated by the applicating person. However, it may also be possible for the number of successive active-medium pulses to be predetermined or settable.

In particular, the working pressure p_{A} can be set on the device 1. In burst mode, the number of times the injection valve 20 injects the working medium AM into the pressure volume 21 can also be set. The working pressure p_{A} (in relation to the initial pressure in the pressure volume 21) particularly influences the pressure and speed of the active medium WM ejected from the nozzle unit 23, but also the injection quantity E. The time interval between successive injection processes with the injection valve 20 could also be settable in burst mode. In burst mode, the temporal length and frequency of the active-medium pulses, and possibly also the quantity of active medium WM emitted, can be set. The settings can also be made depending upon a desired injection volume. Patient characteristics, such as skin type, age, or injection site, could also be taken into account.

In burst mode, the device 1 can also be operated in such a way that active medium WM is delivered from the nozzle unit 23 at predetermined intervals-for example, as long as a control button 30 is actuated, e.g., via a control button 30 and/or a safety switch 31. It is therefore up to the applicating person to decide how long the active medium WM is injected into the treated body surface. In the preferably used normal mode, however, only one active-medium pulse is triggered when the device 1 is actuated-for example, via a control button 30 and/or a safety switch 31.

An active-medium pulse can also be emitted at a modulated pressure, as shown in FIG. 7. FIG. 7 shows an example of the pressure curve in the pressure volume 21 with an active-medium pulse over the time T, starting at the initial pressure in the pressure volume 21. The pressure curve corresponds to the injection pressure p_{E}, i.e., the pressure at which the working medium AM is injected into the pressure volume 21 by the injection valve 20. The dashed line shows an example of the pressure curve without pressure modulation. With pressure modulation, the pressure in the pressure volume 21 is varied during the active-medium pulse. The pressure modulation naturally also modulates the injection quantity E, emitted by the nozzle unit 23 during an active-medium pulse, and the pressure of the active-medium pulse.

In the exemplary embodiment example shown in FIG. 7, working medium AM is first injected into the pressure volume 21 at a first injection pressure p_{E1} for a first time period T1—for example, 2 to 5 ms. This is followed by a second time period T2, e.g., 2 to 40 ms, in which working medium AM is injected at a second injection pressure p_{E2}, wherein the second injection pressure p_{E2} is lower than the first injection pressure p_{E1}. With such pressure modulation, for example, the first high injection pressure p_{A1} can be used to penetrate the body surface-for example, the epidermis. With the lower second injection pressure p_{A2}, a depot of the active medium WM can then be created under the body surface-for example, in the deeper skin layer.

With pressure modulation, other pressure curves are of course also conceivable and possible. A desired pressure curve can be set for each active-medium pulse, e.g., in burst mode, or only at specific intervals if required. It is also possible to combine different pressure curves in successive active-media pulses. A combination of active-medium pulses with and without pressure modulation is also possible.

The pressure modulation can be realized with the injection valve 20. The applicator B is supplied with working medium AM at a specific working pressure p_{A}. This pressure is present at the inlet 27 of the injection valve 20. This is the maximum injection pressure p_{E}, that can be achieved in the pressure volume 21 (apart from losses). This maximum pressure is set when the injection valve 20 is fully opened. The inlet pressure at the injection valve 20 is applied to the outlet of the injection valve 20 in a short time, typically in the range of a millisecond, which is indicated by the ramp-like edges in FIG. 7. If, on the other hand, the injection valve 20 is not fully opened, but only partially, a throttling is effected which results in a lower pressure being present at the outlet 28 of the injection valve 20 (apart from additional losses in the injection valve 20 for throttling) than at the inlet 27, and thus the injection pressure p_{E} into the pressure volume 21 is lower. This correlation between this throttling of the injection pressure p_{E} and the extent of the opening (e.g., as a percentage of the maximum opening) can in turn be determined experimentally and can be assumed to be known and can also be stored in the device 1—for example, in the control unit 16. A desired injection pressure p_{E} can thus be set via the extent of the opening of the injection valve 20—for example, by the control unit 16 as a function of a desired pressure curve.

Pressure modulation is thus characterized by the fact that the injection pressure p_{E} of the working medium AM into the pressure volume 21 is varied during an active-medium pulse. This is done advantageously with the injection valve 20 by controlling the opening position of the injection valve 20.

The described structure of the applicator B ensures complete separation of the working medium AM and the active medium WM in the nozzle unit 23. Any contamination of the active medium WM in the nozzle unit 23 by the high-pressure side of the device 1 with the working medium AM is physically impossible.

For needleless injection of the active medium WM, the nozzle unit 23 can be placed with the at least one nozzle opening 29 directly on the patient's body surface to be treated, or placed at a freely selectable distance from the body surface to be treated. In the latter case, to avoid media splashes, it is advantageous to provide on the nozzle unit 23 a tubular spacer 41, which surrounds the media jet emitted by the nozzle unit 23 (see FIG. 4 for an exemplary embodiment of such a spacer 41). The spacer 41 can also be used to maintain a specific distance from the patient's body surface.

It has been found to be advantageous for a single-hole nozzle with a nozzle opening 29 if the distance between the nozzle opening 29 and the patient's body surface is between 2 and 25 mm, preferably 5 and 15 mm. This can be easily ensured by a spacer 41. In this case, the spacer 41 is placed directly on the patient's body surface.

When using a multi-hole nozzle with several nozzle openings 29, a spacer 41 with a conical inner bore has proven to be advantageous. Cone angles of the inner bore between 25° and 35° have proven successful in studies. The distance from the nozzle opening 29 to the patient's body surface preferably varies between 15 and 40 mm, depending upon the cone angle.

In the case of a single-hole nozzle, the spacer 41 can also have a conical inner bore, wherein a cone angle of less than 5° is sufficient.

To simulate microneedling (mesotherapy), a nozzle unit 23 with several nozzle openings 29 is used with small nozzle bores (0.10 to 0.15 mm) and reduced working pressure p_{A} (100-200 bar), and the active medium WM is injected at several points simultaneously only into the superficial layers of the skin. The applicating person can guide the applicator B in a slow movement over the skin surface to be treated, wherein active medium WM is delivered in burst mode at intervals of preferably between 0.25 and 0.5 seconds. In this treatment, a spacer 41 with a length of between 2 and 60 mm, preferably 15 and 50 mm, is preferably used.

A control button 30 can be arranged on the device 1, e.g., on the applicator B as indicated by a dashed line in FIG. 2, e.g., on an applicator housing 32, with which the applicator B is actuated. The control button 30 can be connected to the control unit 16 via the control line 12. When the applicating person presses the control button 30 on the applicator B, the generation of at least one active-medium pulse begins.

However, the control button 30 can also be provided on the base unit A (as in FIG. 1).

For safety reasons, an additional safety switch 31, e.g., a foot switch, can also be provided on the device 1, preferably on the base unit A. Such a switch 31 can be used to ensure that the applicator B generates active-medium pulses only when the safety switch 31 is actuated. Actuation of a control button 30 would therefore have no effect as long as the safety switch 31 is not actuated. During operation of the applicator B, the generation of the active-medium pulses can be terminated immediately if the safety switch 31 is no longer actuated.

A lamp 30a, 31a, e.g., a two-color LED, can also be installed in the control button 30 and/or in the safety switch 31 (as in the embodiment according to FIG. 1) in order to indicate the system status of the device 1. This lamp 30a, 31a lights up, for example, when the device 1 is ready for operation. If the device is not ready for operation, it can be provided that the lamp 30a, 31a not light up, or light up in a different color.

Also for safety reasons, it can be provided that the applicator B only generate active-medium pulses when the control button 30 has been pressed continuously for a predetermined time period-for example, in the range of seconds. This prevents the generation of active-medium pulses if the control button 30 is actuated unintentionally.

The device 1 can be operated in such a way that the pump 3 generates the set working pressure p_{A}, if necessary with the pressure control valve 6. Once the working pressure p_{A} is reached, the pump 3 switches off. The pressure accumulator 10 keeps the working pressure p_{A} approximately constant. Only when the applicator B is actuated for a specific time period, e.g., 1 to 5 s, to generate the active-medium pulses does the pump 3 start up again and generate the set working pressure p_{A} for a set period of time, e.g., 20 to 60 s, before the pump 3 is switched off again. Each new actuation of the applicator B for the specified time period restarts the pump 3. This mode of operation saves energy and reduces wear in the pump 3.

Further advantageous embodiments of an applicator B according to the invention are explained below with reference to FIG. 3. Only the components and functions that have not already been described above with reference to FIGS. 1 and 2 are explained here.

In the applicator B of FIG. 3, a compression spring 39 is arranged, which acts upon the plunger 22, specifically upon the plunger piston 26 of the plunger 22. The plunger 22 with the plunger piston 26, and also the pressure piston 25 during operation, are moved against the spring action of the compression spring 39. The compression spring 39 also ensures that the plunger 22 is pushed back into its initial position (at minimum pressure volume 21) when the applicator B is depressurized-for example, to detach the nozzle unit 23 from the applicator B. The compression spring 39 is tensioned when the applicator B is actuated, i.e., when working medium AM is injected into the pressure volume 21 by the injection valve 20.

The compression spring 39 as shown in FIG. 3 can of course also be provided in an embodiment as in FIG. 2.

In the embodiment shown, the compression spring 39 engages on the side, facing away from the pressure volume 21, of the plunger piston 26. The other end of the compression spring 39 bears upon a part, immovable relative to the plunger 22 and pressure piston 25, of the applicator B, e.g., upon a part of the applicator housing 32 or upon an abutment 47, as in FIG. 3. The abutment 47 of FIG. 3 is designed as a ring that is screwed into the same internal thread into which the nozzle unit 23 is screwed. This allows the compression spring 39 and the plunger 22 to be easily removed after removing the ring.

To move the plunger 22 back to an initial position with minimum pressure volume 21, e.g., by the compression spring 39 or manually, it may be necessary to depressurize the applicator B. This can be done via the return valve 9 and the return line 8. If the injection valve 20 is opened when the applicator B is depressurized and the plunger 22 is pressed in the direction of the initial position, the working medium AM is fed back into the base unit A via the pressure hose 18 and flows there into the storage tank 2 via the return valve 9 and the return line 8 when the pump 3 is switched off. In this procedure, the injection valve 20 is preferably opened in burst mode in order to reduce the risk of the injection valve 20 overheating due to the long opening time and thus constant energization.

A drainage opening 38 can be provided in the applicator housing 32 in order to easily drain any leakage of working medium AM from the applicator B. However, this drainage opening 38 also serves to immediately indicate a leak in the applicator B. In the event of a leak, the applicator B can be replaced immediately.

In the applicator B, a vent opening 36 can also be provided, which is connected to the pressure volume 21 and can be closed by a vent screw 35. For example, the pressure volume 21 can be filled with working medium AM via the vent opening 36, wherein it is preferable to ensure that the pressure volume 21 is completely vented during the filling process. The vent opening 36 with the vent screw 35, as shown in FIG. 3, can of course also be provided in an embodiment as in FIG. 2.

An additional storage volume 37 for working medium AM can be provided in the region of the inlet 27 of the injection valve 20 and filled with working medium AM at working pressure p_{A} when the applicator B is in operation. For example, the pressure hose 18 can be connected to the storage volume 37, and the inlet 27 can be connected to the storage volume 37. The storage volume 37 ensures that the working pressure p_{A} does not drop when the injection valve 20 is opened, but remains as constant as possible. It has proven to be sufficient if the storage volume 37 corresponds to between ten and one hundred times the injection quantity of the injection valve 20. The storage volume 37 as shown in FIG. 3 can of course also be provided in an embodiment as in FIG. 2 FIG. 4 shows a nozzle unit 23 in detail. In this embodiment, the active medium WM is filled in an active-medium cartridge 40, which is inserted in an inner recess 42 of a cartridge holder 43. This allows the active medium WM to be replaced quickly by replacing the active-medium cartridge 40. To do this, the nozzle unit 23 is detached from the applicator B, e.g., via a thread 44 on the cartridge holder 43, and the active-medium cartridge 40 is replaced. The nozzle unit 23 can then be reattached to the applicator B.

In this embodiment, the active-medium cartridge 40 forms the nozzle chamber 24 of the nozzle unit 23, in which the pressure piston 25 is arranged. In this embodiment, the spacer 41 is also provided on the active-medium cartridge 40. Alternatively, this could also be provided on the cartridge holder 43.

It can also be seen that the pressure piston 25 at the end facing the nozzle opening 29 is shaped in the opposite direction to the inner contour of the active-medium cartridge 40. This allows the active-medium cartridge 40 to be emptied as completely as possible. This is of course also advantageous in an embodiment of the nozzle unit 23 as shown in FIG. 2.

The active-medium cartridge 40 can, at the end (which in use faces the plunger 22) facing away from the nozzle opening 29, be closed with a non-removable plug 45 with a center recess for the plunger 22.

To effectively prevent unwanted, multiple filling of the active-medium cartridge 40, the pressure piston 25 can be provided with at least one sharp-edged, radially protruding lug on its surface. If necessary, the lug can also be arranged on the pressure piston 25 after the filling process. When the pressure piston 25 is moved during operation, such a lug draws a deep groove in the axial direction into the inside of the active-medium cartridge 40, which effectively prevents subsequent pressure build-up in the active-medium cartridge 40 and reuse of the active-medium cartridge 40. Such an active-medium cartridge 40 would be unusable for further use.

It is also advantageous if the active-medium cartridge 40 is arranged in the cartridge holder 43 with as few gaps as possible. In the event of a gap between the active-medium cartridge 40 and the cartridge holder 43, the active-medium cartridge 40 could expand or deform radially, during pressure build-up, due to actuation of the pressure piston 25, which would adversely affect the pressure build-up.

It is therefore advantageous if the inner recess 42 of the cartridge holder 43 is slightly conical in the direction of the nozzle opening 26 (as shown in FIG. 5, for example). A cone angle of between 0.1° and 2°, preferably between 0.15-1°, can be provided. The active-media cartridge 40 has the same cone angle on the outer diameter. This ensures that the entire surface of the active-medium cartridge 40 is in contact with the inner recess 42 of the cartridge holder 43. This can prevent expansion under the internal pressure generated by the pressure piston 25.

In addition, the active-medium cartridge 40 advantageously protrudes axially from the cartridge holder 43 at the end opposite the nozzle opening 29. In one possible embodiment, the active-medium cartridge 40 has a protruding collar 46 at this end. If the nozzle unit 23 with the active-medium cartridge 40 arranged in it is attached to the applicator B, e.g., screwed in, the cartridge, via the axially protruding end, which comes to bear upon the abutment 47 in the applicator B, for example, is pressed into the cartridge holder 43 without play and with a slight press fit. This prevents expansion under the internal pressure generated by the pressure piston 25.

After use, the pressed-in active-medium cartridge 40 can be ejected from the cartridge holder 43 using a special ejection tool.

FIG. 5 shows a further advantageous embodiment of a nozzle unit 23 with a cartridge holder 43 and an active-medium cartridge 40. However, the embodiment described below is just as applicable if the active medium WM is used without active-medium cartridge 40 (for example, as in FIG. 2). This embodiment is particularly advantageous for highly viscous active media WM.

With highly viscous active media WM, it is possible that trapped air does not escape through the nozzle opening 29 during the filling process, but remains in the nozzle chamber 24. Trapped air in the nozzle chamber 24 can adversely affect the pressure build-up in the nozzle unit 23 and should therefore be avoided.

A pressure piston 25 with an axially continuous hole 54 is therefore used. Before the filling process, the pressure piston 25 is pressed all the way down so that the end, facing the nozzle opening 29, of the pressure piston 25 rests axially in the active-medium cartridge 40. At the opposite axial end, a filling connection 51 is provided on the pressure piston 25, to which a filling element (not shown) can be connected for filling. The filling connection 51 can be designed as a fastening thread or preferably as a male luer lock connection. For filling, the filling element can be connected to the hole 54 via the filling connection 51, e.g., by attaching a female luer lock connection of the filling element to the male luer lock connection, or by screwing a connection of the filling element onto the fastening thread. The (highly viscous) active medium WM is pressed through the hole 54 into the active-medium cartridge 40 by this (luer lock) connection of the filling element, wherein the pressure piston 25 in the active-medium cartridge 40 rises. Once the intended filling volume has been reached, the (luer lock) connection of the filling element is removed. The hole 54 can then be closed, e.g., with a plug 53 made of biocompatible elastomer. The nozzle opening 29 can be closed as required during filling.

In this embodiment of the pressure piston 25, the radially protruding lug described above can be placed on the pressure piston 25 after the filling process.

In the embodiment shown in FIG. 5, the hole 54 is widened at the end, facing the plunger 22, of the pressure piston 25. A plug 53, preferably made of biocompatible elastomer, is inserted into this widened part of the hole 54, and the hole 54 is closed. The plunger 22 is stepped at the end facing the pressure piston 25, wherein the reduced end 55 of the plunger 22 moves into the widened part of the hole 54 when the nozzle unit 23 is used and presses the plug 53 into the hole 54 until the formed shoulder 56 of the plunger 22 bears upon the pressure piston 25. This closes the hole 54 securely and liquid-tight, which increases operational safety.

To ensure the sterility of the active medium WM filled into the active-medium cartridge 40, it may be provided that the active-medium cartridge 40 be sterilely covered and sealed on the nozzle side with a closure-for example, by a peel-off foil. The nozzle side of the active-medium cartridge 40, e.g., in the region of the spacer 41, can also be designed as a male luer lock connection, on which a female luer lock plug is arranged. The closure, e.g., the peel-off foil or the luer lock plug, must be removed before inserting the active-medium cartridge 40 into the cartridge holder 43. When the active-medium cartridge 40 is properly inserted into the cartridge holder 43, contact between the active-medium cartridge 40, in particular the nozzle tip, and the non-sterile inner wall of the cartridge holder 43 is prevented by the guidance of the active-medium cartridge 40 in the cartridge holder 43.

An active-medium cartridge 40 with pressure piston 25 as described with reference to FIGS. 4 and 5 is regarded as an independent invention.

Similarly, a nozzle unit 23 with cartridge holder 43 and active-medium cartridge 40 as described with reference to FIGS. 4 and 5 is regarded as an independent invention. Such a nozzle unit 23 is characterized in particular by the fact that the active-medium cartridge 40 is inserted with a press fit in the cartridge holder 43, specifically in the inner recess 42 of the cartridge holder 43, in order to prevent play between the active-medium cartridge 40 and the inner recess 42 in the cartridge holder 43.

1. An applicator for needleless injection of an injectable active medium into a body surface, having a nozzle unit with a nozzle volume into which the injectable active medium is filled, wherein, on the nozzle unit, at least one nozzle opening is provided, which is connected to the nozzle volume, and wherein, in the nozzle unit, a pressure piston is arranged, which at least partially delimits the nozzle volume and which is movably arranged in the nozzle unit, wherein an injection valve is provided in the applicator and a working medium at a working pressure can be applied at an inlet of the injection valve, wherein a pressure volume is provided in the applicator, and the pressure volume is filled with working medium at an initial pressure less than the working pressure, wherein the operational injection valve is configured to inject into the pressure volume a predetermined injection quantity of the working medium at an injection pressure between the working pressure and the initial pressure, wherein there is provided in the applicator a movably arranged plunger, which at a first end has a plunger piston that at least partially delimits the pressure volume, and which, with an opposite second end, bears upon the pressure piston such that the operational plunger is movable in the direction of the pressure piston in order to move the pressure piston so as to deliver an active-medium pulse from the at least one nozzle opening.

2. The applicator according to claim 1, wherein the nozzle unit is detachably arranged on the applicator.

3. The applicator according to claim 2, wherein the nozzle unit is designed with a cartridge holder with an internal recess and an active-medium cartridge is interchangeably inserted in the internal recess, wherein the at least one nozzle opening is provided on the active-medium cartridge and the pressure piston is movably arranged in the active-medium cartridge.

4. The applicator according to claim 2, wherein a tubular spacer is provided on the active-medium cartridge in the region of the nozzle opening, which spacer projects axially from the active-medium cartridge by a predetermined length and at least partially surrounds the nozzle opening, so that the active-medium pulse exits through the tubular spacer.

5. The applicator according to claim 3, wherein the inner recess in the cartridge holder and the active-media cartridge are designed to converge in a conical shape in the direction of the at least one nozzle opening.

6. The applicator according to claim 1, wherein the pressure piston is designed with an axially continuous recess.

7. The applicator according to claim 6, wherein a filling connection is provided on the side, facing away from the at least one nozzle opening, of the pressure piston in order to fill active medium (WM) into the nozzle volume via the filling connection and the axially continuous recess.

8. The applicator according to claim 6, wherein the axially continuous recess is closed with a sealing plug when the active-medium cartridge is filled.

9. The applicator according to claim 1, wherein a control button is provided on the applicator in order to actuate the injection valve.

10. The applicator according to claim 1, wherein the operational injection valve is configured to inject into the pressure volume a predetermined injection quantity of the working medium multiple times at an injection pressure between the working pressure and the initial pressure.

11. The applicator according to claim 10, wherein multiple injections are carried out at predetermined intervals.

12. The applicator according to claim 1, wherein the injection valve varies the injection pressure during the delivery of at least one active-medium pulse.

13. The applicator according to claim 12, wherein the injection valve first injects at a first injection pressure for a first period of time and then injects at a second injection pressure for a second period of time, wherein the second injection pressure is lower than the first injection pressure.

14. The applicator according to claim 1, wherein, in the applicator, a compression spring is arranged, which acts upon the plunger, preferably upon the plunger piston of the plunger.

15. The applicator according to claim 14, wherein the compression spring engages with a first end on the side, facing away from the pressure volume, of the plunger piston, and a second end of the compression spring bears upon a part, immovable relative to the plunger, of the applicator.

16. A device for needleless injection of an injectable active medium into a body surface with an applicator according to claim 1, wherein a base unit is provided which supplies the working medium at the working pressure, and the base unit is connected to the applicator via a pressure hose in order to supply the working medium at the working pressure to the applicator.

17. The device according to claim 16, wherein a pump is provided in the base unit, which increases the pressure of a working medium from a storage tank to the working pressure, wherein the pump delivers into a pressure line that is connected to the pressure hose.

18. The device according to claim 17, wherein, downstream of the pump in the pressure line, a return line branches off, in which a controllable return valve is arranged in order to depressurize the pressure line via the return line when the return valve is open.

19. The device according to claim 16, wherein a safety switch is provided on the device to enable or disable operation of the applicator.

20. A method for needleless injection of an injectable active medium into a body surface using an applicator with a nozzle unit with a nozzle volume containing the active medium, wherein a pressure piston, which at least partially delimits the nozzle volume, is moved in the nozzle unit for discharging the active medium via at least one nozzle opening of the nozzle unit, wherein an injection valve is supplied with a working medium at a working pressure, and the injection valve is injected a predetermined quantity of the working medium at an injection pressure into a pressure volume filled with working medium in the applicator, in which an initial pressure lower than the working pressure acts, and the injection pressure is selected between the working pressure and the initial pressure, and in wherein, by injecting the working medium into the pressure volume, a plunger, arranged movably in the applicator, which at least partially delimits the pressure volume with a plunger piston arranged at a first end and bears upon the pressure piston with an opposite second end, is moved in the direction of the pressure piston, so that an active-medium pulse is emitted from the at least one nozzle opening as a result of the movement of the pressure piston.

21. The method according to claim 20, wherein injecting takes place several times into the pressure volume with the injection valve in order to emit several active-media pulses.

22. The method according to claim 20, wherein the injection valve varies the injection pressure during at least one active-medium pulse.

23. The applicator according to claim 3, wherein the nozzle opening on the active medium cartridge comprises a Luer lock connection.
